(19) United States
(12) Patent Application Publication
(10) Pub. No.: US 2017/0333446 A1
(43) Pub. Date: Nov. 23, 2017

(54) COMPOUNDED FORMULA FOR MENOPAUSE RELIEF

(71) Applicant: Joseph Rosado, Sanford, FL (US)

(72) Inventor: Joseph Rosado, Sanford, FL (US)

(21) Appl. No.: 15/160,022

(22) Filed: May 20, 2016

(51) Int. Cl. A61K 31/57; A61K 31/566; A61K 31/565

(52) U.S. Cl. CPC A61K 31/57; A61K 31/565; A61K 31/566

(57) ABSTRACT

A sub-prescription strength composition of progesterone, estriol, and dehydroepiandrosterone to treat menopause symptoms administered by a topical cream.

### RELATED APPLICATIONS

None.

### BACKGROUND OF THE INVENTION

WIPO patent application WO2008089405A1 discloses:
“In a further aspect of the invention, the vaginal cream compositions contain about the following levels of hormones (per cc of vaginal cream base): estriol (1-2 mg), estradiol (0-1 mg), progesterone (100-400 mg), and testosterone (0.5-2 mg).”

U.S. Pat. No. 6,967,194 discloses:
a. a ratio of pregnenolone to progesterone of 40 mg/dl to <1.5 ng/ml;
b. a ratio of progesterone to corticosterone and aldosterone of <1.5 ng/ml;
c. a ratio of progesterone to Cortisol of <1.5 ng/ml to 6-23 mcg/dl;
d. a ratio of pregnenolone to DHEA of 35-430 ug/dl to 0.7-3.1 ng/dl;
e. a ratio of DHEA to androstenedione of 35-430 ug/dl to 0.7-3.1 ng/ml;
f. a ratio of androstenedione to testosterone of 0.7-3.1 to 6-86 ng/dl;
g. a ratio of estrone to estriol of 25-75 pg/ml to <1.2;
h. a ratio of androstenedione to testosterone 0.7-3.1 ng/ml to 6-86 ng/dl;
i. a ratio of testosterone to estradiol of 6-86 ng/dl to 25-75 pg/ml; and
j. a ratio of testosterone to estriol of 6-86 ng/dl to <1.2.

PCT Patent Application number PCT/US2008/051431, discloses a vaginal cream compositions containing hormones. More particularly, to a hormonal vaginal cream compositions having combinations and ratios of hormones that are beneficial to menopausal women.

There is also a need for a menopause treatment that consists essentially of a group from progesterone, estriol, and dehydroepiandrosterone (DHEA).

There is a need for a non-pharmaceutical composition that relieves the following menopause symptoms; hot flashes, night sweats, insomnia, decreased libido, irritability, incontinence, vaginal dryness, urinary complaints, repeated urinary tract infection, urgency, hesitancy, frequency, and stress incontinence.

There is a need for a topically applied composition that can reduce any toxicity associated with synthetic oral hormones, which also eliminates or reduces liver toxicity.

### SUMMARY OF THE INVENTION

One aspect is a composition for treating menopause symptoms, comprising: a cream containing about 40 mg/ml of progesterone; about 2.5 mg/ml estriol; and about 50 mg/ml of dehydroepiandrosterone.

A second aspect of the present invention is a method of making a menopause treatment cream, comprising the steps: heating of water soluble ingredients (300); heating of oil soluble ingredients (310); mixing said water soluble ingredients with said oil soluble ingredients (320) in one vessel, then adding progesterone, estriol, and dehydroepiandrosterone (330)

### BRIEF DESCRIPTION OF THE DRAWINGS

FIG. 1 represents an embodiment of the steps of making the invention.

FIG. 2 discloses a composition of an embodiment of the invention.

### DETAILED DESCRIPTION

### Testimonials

Mia M. My name is Mia M. and I am 52 years old. After going through the necessary tests and determining what needed to be in the prescription, I started Hormone Therapy. This Hormone Therapy is completely natural and in no way synthetic. I am 52 years old now and I don't have any of those feelings that all my friends and family members have at this age like mood swings and sweats from peri-menopause or even menopause. I feel young again and am enjoying my life to the fullest. I never thought I would ever feel like this again. I am so thankful every day that I was led to Dr. Rosado. The Hormone Therapy he has me on has changed my life forever, and I know that I don't have to feel old, depressed, tired, frustrated, and negative anymore.

Kathleen E. I wanted to let you know that the new formulations that you put me on this time around have been LIFE CHANGING!!! Sexual interest and pleasure has not only RETURNED but is BETTER than ever before . . . I am still marveling over the change . . . :) We are back to having sex twice a week without thinking about it—which is a BIG improvement over 1-2 a YEAR!!!!

### Kathleen E.

### REFERENCE NUMERALS

10 compound formula for menopause relief
20 compounded formula
30 progesterone
40 estriol
50 dehydroepiandrosterone (DHEA)
60 dosages
80 cream
90 triple active preparation formulation
100 oil-in-water emulsion cream
110 prescribed levels
120 water (70-90%)
130 botanical extracts (1-10%)
140 nut oils (1-10%)
150 vegetable and seed oils and butter (1-15%)
160 progesterone (1-8%)
170 dehydroepiandrosterone (DHEA) (1-5%)
180 estriol (0-0.3%)
190 plant derived extracts and exudates (1-10%)
200 plant derived polyol (1-10%)
210 vegetable oil derived fatty acids (1-5%)
220 vegetable oil derived fatty alcohols (1-5%)
230 vegetable oil derived fatty ester (1-10%)
240 plant derived natural emulsifier (1-5%)
250 animal derived natural emulsifier (1-5%)
260 Oil Soluble Vitamins (0-2%)
270 water soluble vitamins (0-5%)
280 other Inactive (0-1%)
300 heating water soluble ingredients
310 heating oil soluble ingredients
320 mixing the water soluble and oil soluble ingredients
330 adding progesterone, estriol, and dehydroepiandrosterone

### Using the Invention

Various non-pharmaceutical acceptable excipients can be used. In some embodiments, the pharmaceutically acceptable excipient can be, but is not limited to, a stiffening agent, an oil, a solvent, an emulsifier, a humectant, a buffering agent, a filler, an emollient, a stabilizer, or combinations thereof.

The invention herein may be referred to as a compounded formula for menopause relief 10.

In one embodiment the herein invention, which may be referred to herein as a compound formula for menopause relief 10, may be a compounded formula 20 as a topical cream 80, which can be used for the management and resolution of menopause related signs and symptoms 10; including but not limited to hot flashes, vaginal dryness, mood swings, short term memory loss, decreased libido, etc. 10.

In one embodiment, the current invention 10 may be a compounded formula 20 or blend 20 composed of a combination of three substances, progesterone 30, estriol 40 and dehydroepiandrosterone (DHEA) 50 in dosages 60 that are below prescription strength. In this case prescription strength means amounts about equal to over the counter supplemental dosages, which is about 20 mg of progesterone 30, about 1.25 mg of estriol 40, and about 25 mg of DHEA 50.

An alternative embodiment includes about 40 mg/ml of progesterone 30, about 2.5 mg/ml of estriol 40, and about 50 mg/ml of DHEA 50, in cream 80 form be applied topically 0.5 ml twice per day. It is best to apply this cream 80 on the body where the skin is thin and sensitive such as the neck, décolleté area, upper chest, breast, under arms, tummy, and inner thighs.

A present embodiment 10 may also include a triple active preparation formulation 90. In one embodiment 10 the topical preparation 80, also referred to as cream 80 may be in the form of an oil-in-water cream emulsion 100. This oil-in-water emulsion cream 100 contains prescribed levels 110 of the three active agents; about 40 mg per half ml of progesterone 30, about 25 mg per one half ml of dehydroepiandrosterone (DHEA) 50, and about 1.25 mg per half ml.

Another embodiment of an oil-in-water emulsion cream 100 contains prescribed levels 110 of the three active agents; about 20 mg per half ml of progesterone 30, about 25 mg per one half ml of dehydroepiandrosterone (DHEA) 50, and about 1.25 mg per half ml.

The oil-in-water emulsion cream 100 may be applied topically on the skin.

### Making the Invention

During the course of formulation of this preparation, the final preparation must be pleasing to the consumer and the user, stable, functional and reproducible. In order to insure product safety certain tests and clinical studies must additionally be performed.

The preparation obtained by this invention is smooth, creamy, white, practically odorless which spreads easily on the skin and leaves the skin smooth, supple, rehydrated, and emollient.

The preparation has been designed that it has dermal penetration potential. This is performed by normal absorption and blood circulation throughout the skin. Once in the subdermal layer of the skin, it delivers its contents of active agents into the tissue area where it is slowly absorbed through circulation into the blood stream and eventually to the target sites.

This preparation may be prescribed for topical application; In this case the cream is ascertained to exhibit no irritation, no break down into auxiliary molecules that can neither produce any undesirable products nor side effects.

This combination of multiple ingredients; active and inactive may be totally inert to each other as any reactivity may produce undesirable odor, side effects, taste, or discoloration. The following is a general statement of the formula with the specific ingredients used as well as the general categories from which each ingredient has been selected. Other ingredients in the specified categories may also be used once selection is made and initial testing and evaluation have been completed.

The composition contains the following ingredients and or the categories of ingredients in the use range recommend:

Water (70-90%) 120: water 120 must be deionized, demineralized and or purified. The purification method recommend is reverse osmosis (R0);

Botanical extracts (1-10%) 130: Many members of this category of botanical extracts 130 can be used however, the following ones are recommended. Mexican wild yam, red clover leaf, Siberian ginseng root, burdock root, black cohosh, chamomile, milk thistle, comfrey root, cramp bark, rosemary, grape fruit seed and grape seed. These extracts are hydrous (water based), hydroalcoholic (prepared in a base of water and ethanol), hydroglycolic (prepared in a base of water and propylene glycol USP) or hydroglycerol (prepared in a base of water and glycerin Kosher USP). The extracts are prepared using whole leaves, powdered seeds or bark, cut-and-sift roots, rhizome or stems of the specified herb or plant;

nut oils (1-10%) 140: coconut and hazel nut;

vegetable and seed oils and butter (1-15%) 150: shea butter, grape seed, grape fruit seed, carrot seed, sesame seed, olive, avocado, lemon grass, safflower and sun flower;

progesterone (1-8%) 160: micronized powder USP (pharmaceutical grade).

dehydroepiandrosterone (DHEA) (1-5%) 170: micronized powder USP (pharmaceutical grade);

estriol (0-0.3%) 180: micronized powder USP (pharmaceutical grade).

plant derived extracts and exudates (1-10%) 190: aloe vera lenne (aloe barbadensis miller);

plant derived polyol (1-10%) 200: glycerin USP;

vegetable oil derived fatty acids (1-5%) 210: linoleic, linolenic, stearic acid, palmitic acid and lauric acid;

vegetable oil derived fatty alcohols (1-5%) 220: cetyl and cetearyl;

vegetable oil derived fatty ester (1-10%) 230: glyceryl stearate;

plant derived natural emulsifier (1-5%) 240: lecithin from soya beans;

animal derived natural emulsifier (1-5%) 250: lecithin from eggs; and

oil soluble vitamins (0-2%) 260: vitamins A, E or K.

Water soluble vitamins (0-5%) 270: thiamine, riboflavin, niacin or nicotinic acid, folic acid and dexapanthenol; and

other inactive (0-1%) 280: allantoin.

The emulsion may be represented by the following formulation (the below are ratios):

Purified water 25.3, avocado oil 5.0, sesame seed oil 5.0, olive oil 5.0, progesterone USP micronized 8.0, dehydroepiandrosterone (DHEA) micronized powder USP (pharmaceutical grade) 5.0, estriol micronized powder USP (pharmaceutical grade) 2.5, aloe vera gel (aloe barbadensis miller) 10.0, red clover extract 2.0, burdock root cohosh extract 2.0, chamomile extract 2.0, Siberian ginseng root extract 2.0, shea butter 2.0, evening primrose oil 0.5, vitamin E acetate 1.0, MSM (methyl sulfonyl methane) 2.0, soy lecithin 2.5, vegetable glycerin 5.0, milk thistle extract 2.0, stearic acid triple pressed 3.5, cetyl alcohol 2.0, cetearyl alcohol (and) ceteareth −20 3.0, dexapanthenol (vitamin B-5) 1.5, allantoin 0.2, grape seed extract 0.5 and phenoxyethanol 0.5.

All ingredients are weighted and placed in a designated vessel. The ingredients are grouped into two categories based on solubility; water soluble group and oil soluble group.

In another embodiment, the emulsion may be represented by the following formulation (the below are ratios):

Water soluble ingredients: purified water 25.3, aloe vera gel (aloe barbadensis miller) 10.0, red clover extract 2.0, burdock root cohosh extract 2.0, chamomile extract 2.0, Siberian ginseng root extract 2.0, MSM (Methyl Sulfonyl Methane) 2.0, vegetable glycerin 5.0, milk thistle extract 2.0, dexapanthenol (vitamin B-5) 1.5, allantoin 0.2, grape seed extract 0.5 and phenoxyethanol 0.5.)

Oil phase consists of all the oil soluble ingredients: avocado Oil 5.0, sesame seed oil 5.0, olive oil 5.0, shea butter 2.0, evening primrose oil 0.5, vitamin E acetate 1.0, soy lecithin 2.5, stearic acid triple pressed 3.5, cetyl alcohol 2.0, cetearyl alcohol (and) ceteareth −20 3.0

Active ingredients: progesterone USP micronized 8.0, dehydroepiandrosterone (DHEA) micronized powder USP (pharmaceutical grade) 5.0, estriol micronized powder USP (pharmaceutical grade) 2.5.

The water soluble and oil phase groups of ingredients will be heated in a separate stainless steel (SS) vessel to 80 degrees Celsius. The two hot phases are then discharged together in the larger container of the two or into a third container with the capacity of the total batch. Once the two phases are mixed together in one vessel, the three active ingredients are added with high sheer to thus forming emulsion at 50 degrees Celsius. The duration of heating is not important. It is important that the mixture reached the temperature with agitation.

In another embodiment, the water phase consists of all the water soluble ingredients: purified Water 25.3, progesterone USP micronized 8.0, dehydroepiandrosterone (DHEA) micronized powder USP (pharmaceutical grade) 5.0, estriol micronized powder USP (pharmaceutical grade) 2.5, aloe vera gel (aloe barbadensis miller) 10.0, red clover extract 2.0, burdock root cohosh extract 2.0, chamomile extract 2.0, Siberian ginseng root extract 2.0, MSM (Methyl Sulfonyl Methane) 2.0, vegetable glycerin 5.0, milk thistle extract 2.0, dexapanthenol (vitamin B-5) 1.5, allantoin 0.2, grape seed extract 0.5 and phenoxyethanol 0.5.).”

The oil phase consists of all the oil soluble ingredients: avocado oil 5.0, sesame seed oil 5.0, olive oil 5.0, shea butter 2.0, evening primrose oil 0.5, vitamin E acetate 1.0, soy lecithin 2.5, stearic acid triple pressed 3.5, cetyl alcohol 2.0, cetearyl alcohol (and) ceteareth −20 3.0.

High sheer mixing is applied and continuous until cream is uniformed. Sample of the cream is taken to the laboratory for identification and testing for physical properties such as Description of Physical form, Odor, pH, Specific Gravity and Viscosity at ambient. A sample is sent to an outside laboratory to essay the active ingredients results are accepted when falling in the range of (+/−) 1-5% plus or minus.

It is to be noticed that the three active ingredients, (1) progesterone, (2) estriol, and (3) DHEA are not to be mixed with neither the water phase, nor the oil phase.

These ingredients are saved for later addition to the emulsion, prior to filling. Equipment used for the manufacturing and packaging of this oil-water cream emulsion formulation are known to those involved in the art. The water phase consists of all the water soluble ingredients being placed in a heating stainless steel kettle. This mixture of ingredients is heated to 70-80 degrees Celsius. The oil soluble ingredients are charged with continuous agitation and heated to 70-80 degrees Celsius in a separate kettle. Once both hot phases are clear, charge the oil phase into the water phase with continuous agitation and high sheer. Start cooling the cream.

The active ingredients may be added at this time when the temperature is dropped to approx. 60-65 degrees Celsius. Continue mixing until smooth and creamy emulsion is obtained. Continue cooling while mixing until product reaches room temperature. Product is now ready for filling.

The following is a list of the Latin/INCI names for the botanicals used. In order to maintain quality, consistency and efficacy, it is recommended that the botanical species specified are to be used. Botanicals may be used in the form of a high quality liquid extract or cut and sift grade of botanicals to be used in the manufacture of a suitable liquid extract; which may be one of the following:

Red Clover Trifolium Pratense Flower Extract;

Burdock Arctium Lappa Root Extract;

Cohosh Cimicifuga Racemosa Root Extract;

Siberian Ginseng Root Eleutherococcus Senticosus Extract;

Chamomile Chamomilla Recutita (Matricaria) Flower Extract;

Milk Thistle Silybum Marianum Extract; or

Grapeseed Vitis Viniferum Extract.

DHEA, Progesterone and Estriol, along with many other hormones, continue to decline in the human body as it ages. Oral and/or topical supplementation becomes necessary in order to maintain optimal health and the proper function of the various faculties of the body. A cream is designed per this invention to impart moisturization, soothing, smoothness and retexturization of the skin.

The cream is impregnated with clinically effective doses of DHEA, Progesterone and Estriol. The cream is applied topically with certain periodicity. When the cream is applied to skin, it is massaged into the neck, chest and/or thighs. Typical advantages expected of this application include relief from hot flashes, improved mood swings, improved libido and the alleviation of signs of vaginal dryness or atrophy.

Some pre-menopausal females prefer to use progesterone at the end of the menstrual cycle, during which the dose is also reduced. Being the hormone for menstrual symptoms, Estriol is specified in this cream to correct the symptoms mentioned above, in addition to treating several vaginal disorders, including, but not limited to dryness, burning, itching, inflammations and unusual discharges.

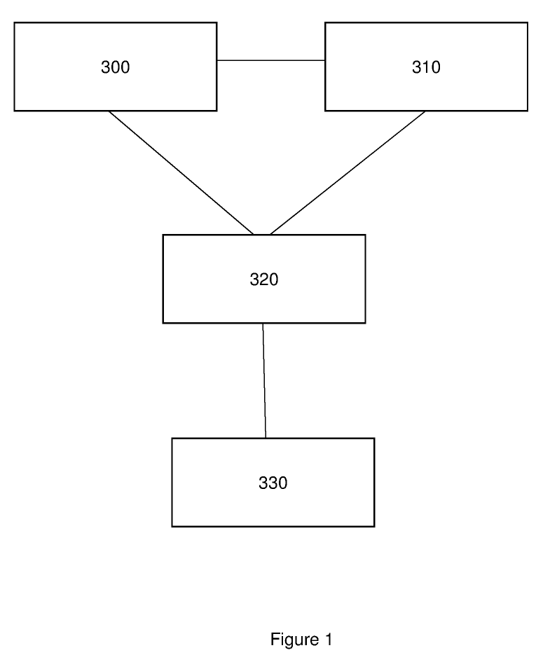

FIG. 1 discloses an embodiment of the steps to make the present invention; which may be heating of water soluble ingredients 300; and heating of oil soluble ingredients 310; then mixing said water soluble ingredients with said oil soluble ingredients 320 in one vessel, then adding progesterone, estriol, and dehydroepiandrosterone 330.

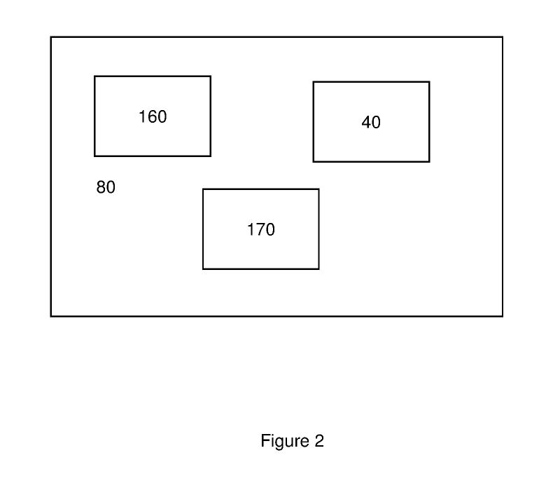

FIG. 2 discloses a composition of an embodiment of the invention.

Accordingly, those skilled in the art will recognize that the unique combinations of ingredients set forth herein are selected so as to be the healthiest and chemically benign components possible for use for the topical cream 80 formulation.

The methods of making and using the present invention are directed towards reducing and minimizing any toxicity or harmful side effects.

Having described the invention with reference to particular compositions and methods of creating the cream 80 it is not intended that the invention be limited by such illustrative embodiments, and that modifications can be made without departing from the scope or spirit of the invention, as defined by the claims. All modifications and variations be included within the scope of the invention.

I claim:

1. A composition for treating menopause symptoms, comprising:
a cream (80) containing about 40 mg/ml of progesterone (160); about 2.5 mg/ml estriol (40); and about 50 mg/ml of dehydroepiandrosterone (170).

2. A method of making a menopause treatment cream, comprising the steps:
heating of water soluble ingredients (300);
heating of oil soluble ingredients (310);
mixing said water soluble ingredients with said oil soluble ingredients (320) in one vessel, then adding progesterone, estriol, and dehydroepiandrosterone (330)

3. A method of making a menopause treatment of claim 2, further comprising the steps of:
said heating of water soluble ingredients (300) to about 80 degrees Celsius;
said heating of oil soluble ingredients (310) to about 80 degrees Celsius separately from said water soluble ingredients;
said mixing said water soluble ingredients with said oil soluble ingredients (320) in one vessel, then adding progesterone, estriol, and dehydroepiandrosterone (330) with high sheer at 50 degrees Celsius.

4. A method of making a menopause treatment of claim 3, wherein said water soluble ingredients (300) are ratios of Purified Water 25.3, Progesterone USP Micronized 8.0, Dehydroepiandrosterone (DHEA) Micronized powder USP (Pharmaceutical Grade) 5.0, Estriol Micronized powder USP (Pharmaceutical Grade) 2.5, Aloe Vera Gel (Aloe barbadensis Miller) 10.0, Red Clover Extract 2.0, Burdock Root Cohosh Extract 2.0, Chamomile Extract 2.0, Siberian Ginseng Root Extract 2.0, MSM (Methyl Sulfonyl Methane) 2.0, Vegetable Glycerine 5.0, Milk Thistle Extract 2.0, Dexapanthenol (vitamin B-5) 1.5, Allantoin 0.2, Grape Seed Extract 0.5 and Phenoxyethanol 0.5.); and
said oil soluble ingredients are ratios of Avocado Oil 5.0, Sesame Seed Oil 5.0, Olive Oil 5.0, Shea Butter 2.0, Evening Primrose Oil 0.5, Vitamin E Acetate 1.0, Soy Lecithin 2.5, Stearic Acid Triple Pressed 3.5, Cetyl Alcohol 2.0, Cetearyl Alcohol and Ceteareth −20 3.0.

5. A method of making a menopause treatment of claim 3, wherein said water soluble ingredients (300) are ratios of Purified Water 25.3, Aloe Vera Gel (Aloe barbadensis Miller) 10.0, Red Clover Extract 2.0, Burdock Root Cohosh Extract 2.0, Chamomile Extract 2.0, Siberian Ginseng Root Extract 2.0, MSM (Methyl Sulfonyl Methane) 2.0, Vegetable Glycerine 5.0, Milk Thistle Extract 2.0, Dexapanthenol (vitamin B-5) 1.5, Allantoin 0.2, Grape Seed Extract 0.5 and Phenoxyethanol 0.5.);
said oil soluble ingredients are ratios of Avocado Oil 5.0, Sesame Seed Oil 5.0, Olive Oil 5.0, Shea Butter 2.0, Evening Primrose Oil 0.5, Vitamin E Acetate 1.0, Soy Lecithin 2.5, Stearic Acid Triple Pressed 3.5, Cetyl Alcohol 2.0, Cetearyl Alcohol and Ceteareth −20 3.0; and after the water soluble ingredients and oil soluble ingredients are mixed at about 80 degrees Celsius, then add;
Progesterone USP Micronized 8.0, Dehydroepiandrosterone (DHEA) Micronized powder USP (Pharmaceutical Grade) 5.0, Estriol Micronized powder USP (Pharmaceutical Grade) 2.5 and mix at high sheer at about 50 degrees Celsius.

6. A method of making a menopause treatment of claim 2, further comprising the steps of:
heating said water soluble ingredients (300) to about 70-80 degrees Celsius;
heating said oil soluble ingredients (310) to about 70-80 degrees Celsius, said oil soluble ingredients are charged with continuous agitation, after said water soluble ingredients and said oil soluble ingredients are clear;
mixing said water soluble ingredients with said oil soluble ingredients (320) with continuous agitation and high sheer; then let this mixture cool;
adding progesterone, estriol, and dehydroepiandrosterone (330) when the temperature is dropped to approx. 60-65 degrees Celsius; and
continue mixing until smooth and creamy emulsion is obtained.

7. A method of making a menopause treatment of claim 6, wherein said water soluble ingredients (300) are ratios of Purified Water 25.3, Progesterone USP Micronized 8.0, Dehydroepiandrosterone (DHEA) Micronized powder USP (Pharmaceutical Grade) 5.0, Estriol Micronized powder USP (Pharmaceutical Grade) 2.5, Aloe Vera Gel (Aloe barbadensis Miller) 10.0, Red Clover Extract 2.0, Burdock Root Cohosh Extract 2.0, Chamomile Extract 2.0, Siberian Ginseng Root Extract 2.0, MSM (Methyl Sulfonyl Methane) 2.0, Vegetable Glycerine 5.0, Milk Thistle Extract 2.0, Dexapanthenol (vitamin B-5) 1.5, Allantoin 0.2, Grape Seed Extract 0.5 and Phenoxyethanol 0.5.); and
said oil soluble ingredients are ratios of Avocado Oil 5.0, Sesame Seed Oil 5.0, Olive Oil 5.0, Shea Butter 2.0, Evening Primrose Oil 0.5, Vitamin E Acetate 1.0, Soy Lecithin 2.5, Stearic Acid Triple Pressed 3.5, Cetyl Alcohol 2.0, Cetearyl Alcohol and Ceteareth −20 3.0.

8. A method of making a menopause treatment of claim 6, wherein said water soluble ingredients (300) are ratios of Purified Water 25.3, Aloe Vera Gel (Aloe barbadensis Miller) 10.0, Red Clover Extract 2.0, Burdock Root Cohosh Extract 2.0, Chamomile Extract 2.0, Siberian Ginseng Root Extract 2.0, MSM (Methyl Sulfonyl Methane) 2.0, Vegetable Glycerine 5.0, Milk Thistle Extract 2.0, Dexapanthenol (vitamin B-5) 1.5, Allantoin 0.2, Grape Seed Extract 0.5 and Phenoxyethanol 0.5.);
said oil soluble ingredients are ratios of Avocado Oil 5.0, Sesame Seed Oil 5.0, Olive Oil 5.0, Shea Butter 2.0, Evening Primrose Oil 0.5, Vitamin E Acetate 1.0, Soy Lecithin 2.5, Stearic Acid Triple Pressed 3.5, Cetyl Alcohol 2.0, Cetearyl Alcohol and Ceteareth −20 3.0; and after the water soluble ingredients and oil soluble ingredients are mixed at about 80 degrees Celsius, then add;
Progesterone USP Micronized 8.0, Dehydroepiandrosterone (DHEA) Micronized powder USP (Pharmaceutical Grade) 5.0, Estriol Micronized powder USP (Pharmaceutical Grade) 2.5 and mix at high sheer at about 50 degrees Celsius.
